(19) United States
(12) Patent Application Publication
(10) Pub. No.: US 2009/0208480 A1
(43) Pub. Date: Aug. 20, 2009

(54) LONG HALF-LIFE RECOMBINANT BUTYRYLCHOLINESTERASE

(76) Inventors: Yue Huang, Vaudreuil-Dorion (CA); Harvey Wilgus, Beaconsfield (CA)

Correspondence Address: Alan J Grant;Carella Byrne Brain Giffillan Cecchi StwartOlstein, 5 Becker Farm, Roseland, NJ, 07068, US

(21) Appl. No.: 12/309,909

(22) PCT Filed: Aug. 2, 2007
(86) PCT No.: PCT/US07/17279

§ 371 Date: Feb. 2, 2009

Related U.S. Application Data
(60) Provisional application No. 60/835,827, filed on Aug. 4, 2006.

(51) Int. Cl. A61K 38/46; C12N 9/18; A61P 39/00

(52) U.S. Cl. 424/94.6; 435/197

(57) ABSTRACT

The present invention provides for butyrylcholinesterase (BChE) attached to polyethylene glycol (PEG) to form a complex having greatly increased mean residence time (MRT) in the system of an animal following injection thereinto. Also disclosed are compositions of such complexes, methods of preparing these complexes and method for using these complexes and compositions in the treatment and/or prevention of toxic effects of poisons, such as neurotoxins, to which said animals, such as humans, have been, or may become, exposed.

This application claims priority of U.S. Provisional Application 60/835,827, filed 4 Aug. 2006, the disclosure of which is hereby incorporated by reference in its entirety.

### FIELD OF THE INVENTION

This invention relates to the chemical modification of butyrylcholinesterase (BChE) by polyethylene glycol (PEG) to improve circulatory mean residence time (MRT) of the protein and reduce protein immunogenicity for pharmaceutical and bio-defense applications.

### BACKGROUND OF THE INVENTION

Use of organophosphate and related compounds as pesticides and in warfare over the last several decades has resulted in a rising number of cases of acute and delayed intoxication, causing damage to the peripheral and central nervous systems and resulting in myopathy, psychosis, general paralysis, and death. Such noxious agents act by inhibiting cholinesterase enzymes and thereby prevent the breakdown of neurotransmitters, such as acetylcholine, causing hyperactivity of the nervous system. For example, build-up of acetylcholine causes continued stimulation of the muscarinic receptor sites (exocrine glands and smooth muscles) and the nicotinic receptor sites (skeletal muscles). In addition, exposure to cholinesterase-inhibiting substances can cause symptoms ranging from mild (e.g., twitching, trembling) to severe (e.g., paralyzed breathing, convulsions), and in extreme cases, death, depending on the type and amount of cholinesterase-inhibiting substances involved. The action of cholinesterase-inhibiting substances such as organophosphates and carbamates makes them very effective as pesticides, such as for controlling insects. When mammals, such as humans, are exposed to these compounds (e.g., by inhalation), they often experience the same negative effects.

The devastating impact of certain cholinesterase-inhibiting substances on humans has led to the development of these compounds as “nerve gases” or chemical warfare agents. Nerve agents are among the most toxic. Such compounds are related to organophosphorus insecticides in that they are both esters of phosphoric acid. Major nerve agents include diisopropylfluorophosphate (DFP), GA (tabun), GB (sarin), GD (soman), CF (cyelosarin), GE, CV, yE, VG (amiton), VM, VR (RVX or Russian VX), VS, and VX.

Organophosphate poisoning is currently treated by intravenous or intramuscular administration of combinations of drugs, including carbamates (e.g., pyridostigmine), anti-muscarinics (e.g., atropine), and ChE-reactivators such pralidoxime chloride (2-PAM, Protopam). One approach has utilized cholinesterase enzymes for the treatment of organophosphate exposure. Post-exposure toxicology can be prevented by pretreatment with cholinesterases, which act to sequester the toxic organophosphates before they reach their physiological targets.

Use of cholinesterases as pre-treatment drugs has been successfully demonstrated in animals, including non-human primates. For example, pretreatment of rhesus monkeys with fetal bovine serum-derived AChE or horse serum-derived BChE protected them against a challenge of two to five times the LD5O of pinacolyl methylphosphonofluoridate (soman), a highly toxic organophophate compound used as a chemical weapon (Broomfield et al., J. Pharmacol. Exp. Ther., 1991, 259:633-638; Wolfe et al., Toxicol, Appl. Pharmacol., 1992, I17(2):189-193). Administration of sufficient exogenous human BChE can protect mice, rats, and monkeys from multiple lethal-dose organophosphate intoxication (See, e.g., Raveh et al., Biochemical Pharmacology, 1993, 42:2465-2474; Raveh et al., Toxicol. Appi. Pharmacol., 1997, 145:43-53; Allon et al, Toxicol. Sei., 1998, 43:121-128). Purified human BChE has been used to treat organophosphate poisoning in humans, with no significant adverse immunological or psychological effects (Cascio et al., Minerva Anestesiol., 1998, 54:337).

Titration of organophosphates both in vitro and in vivo demonstrates a 1:1 stoichiometry between organophosphate-inhibited enzymes and the cumulative dose of the toxic nerve agent.

Modification of pharmaceuticals by polyethylene glycol (PEG) has been reported to improve half-life and reduce immunogenicity. Proteins modified by PEG and approved by the FDA include: ADAGEN® (pegademase bovine) by Enzon, ONCASPA® (Pegaspargase) by Enzon, PEGASYS® (peginterferon alfa-2a) by Roche, PEG-INTRON® (peginterferon alfa-2b) by Schering-Plough and MACUGEN® (pegabtanib) by Eyetech & Pfizer Inc.

PEG can be attached to proteins at a variety of sites, including amino groups, such as those on lysine residues, or at the N-terminus, as well as thiol groups on cysteine, or other reactive groups on the protein surface.

However, PEG modification of proteins, such as enzymes, is known to present some problems such as: 1) non-specific attachment sites, 2) reduction or loss of biologic activities (such as enzyme activity), and 3) outcome of PEGylation is often unpredictable. Ideally, attachment of a PEG to, for example, a protein should increase circulatory time of the drug in an animal, such as a human, as well as reduce immunogenicity and in vivo degradation.

Butyrylcholinesterase (BChE) can be found in nature in the form of monomers, dimers and tetramers. BChE may also be produced by recombinant techniques, including production in transgenic animals. Produced transgenically (referred to by the name PROTEXIA™) BChE is a mixture of dimer and monomer with a small percentage of tetramer. For example, transgenic recombinant BChE secreted in goat's milk is about 80% dimers and 20% monomers (determined by SEC-HPLC chromatography followed by Ellman activity assay of collected fractions).

### BRIEF SUMMARY OF THE INVENTION

In one aspect, the present invention relates to PEGylated (meaning attached to PEG—polyethylene glycol) recombinant butyrylcholinesterase (PEG-BChE), such as is produced in the milk of transgenic goats.

In a specific embodiment, the activated PEG reagents include mono-functional methoxy-activated polymer of succinimidyl derivatives such as succinimidyl propionic acid, α-methylbutanoate, and N-Hydroxysucciminidyl. These reagents facilitate attachment of PEG to the amino groups of the protein.

In a specific and non-limiting embodiment, the activated PEG reagents are mono-functional methoxy-activated polymer bearing aldehyde groups such as Butyraldehydyl-PEG. The N-terminal amino group of the protein is specifically targeted by these reagents.

In another specific and non-limiting embodiment, the activated PEG reagents are mono-functional methoxy-activated PEG with o-pyridylthioester. N-terminal thiol groups (cysteine) is specifically target by these reagents.

In a further specific and non-limiting embodiment, the activated PEG reagents are thiol group specific such as Maleimide coupling PEG. Free thiol group (cysteine) on a protein can be specifically target by these reagents.

In another embodiment; the activated PEG reagents are linked to sialic acid, which facilitates targeting of glycans on BChE.

In other embodiments, the activated PEG reagents can be linear PEG, such as mPEG-SPA, branched PEG, such as mPEG2-NHS, or forked PEG, such as mPEG-MAL2.

In additional embodiments, the product of the invention is a pegylated recombinant BChE having either the native BChE amino acid sequence or a mutated amino acid sequence (the latter retaining substantially the biological activity of native BChE).

In another aspect, the present invention relates to compositions of any of the compounds (i.e., pegylated proteins, such as pegylated BChE) of the invention, preferably wherein such compound is present in a pharmaceutically acceptable carrier and in a therapeutically effective amount. Such compositions will generally comprise an amount of such compound that is not toxic (i.e., an amount that is safe for therapeutic uses).

In specific embodiments, the molecular weight of the activated PEG reagents ranges from 5000 Dalton (D or Da) to 500,000 Dalton. In other specific and non-limiting embodiments, the coupling reaction is carried out in a buffer having a pH from 4 to 11, in one case pH 4 to 10, in another case pH 5 to 10, or pH 6 to 10, or pH 6 to 9, with pH values of about pH 6 or 7 or 8 or 9 being most advantageous. In the methods disclosed herein, the PEG:protein molar ratio in conjugation reaction is from 2 to 500, more specifically from 5 to 400, or from 10 to 300, or from 20 to 200 or from 30 to 100, or from 50 to 100, or from 60 to 90, or from 70 to 90, with a ratio of about 80:1 being advantageous. Also in the methods of the invention, the temperature of the conjugation reaction is from, or from 10° C. to 40° C., or from 15° C. to 30° C., or about 20° C. to 25° C., with about 25° C. being advantageous. In addition, in the methods of the invention the conjugation reaction time is from 10 minutes to 24 hours. Also in the methods of the invention the protein concentration in the conjugation reaction is 0.1 to 10 mg/ml.

In accordance with an embodiment of the present invention, the PEGylation products can be analyzed on SDS-PAGE, SEC-HPLC, or by light scattering. In one embodiment, light scattering shows that a PEG-BChE produced according to the present invention contains a PEG of an average molecular weight of 20 kD. PEG attachment sites can be identified by peptide mapping with mass spectrometry and also by dissecting the pegylated protein, such as by trypsin digestion.

In further embodiments, the activity of PEG-BChE (measured by the Ellman assay) is substantially the same as recombinant BChE so that modification of BChE by PEG does not have any disadvantageous impact on its biological activity.

In accordance with the present invention, the in vivo half-life of PEG-BChE is increased over that of BChE.

In a further aspect, the present invention is directed to a method of treating nerve agent poisoning in a subject comprising providing an effective amount of a nerve agent neutralizing enzyme, preferably PEG-BChE, especially where said agent is delivered systemically, such as by injection. Specific and non-limiting subjects are any animals in need of protection from nerve agents, preferably mammals, most preferably human beings.

Alternatively, PEG-BChE agent is in a liquid form. In a such an embodiment, the PEG-BChE may further comprise an excipient. In a further such embodiment, PEG-BChE is administered with an inhaler or a nebulizer.

In still another embodiment, the PEG-BChE is contained in a dry powder form. In such an embodiment, the nerve agent neutralizing enzyme may further comprise an excipient. In a further embodiment, the nerve agent neutralizing enzyme is administered with an inhaler.

### BRIEF DESCRIPTION OF THE DRAWINGS

FIG. 1 shows SDS-PAGE of PROTEXIA™ both PEGylated (lanes 1 and 2) and non-PEGylated (lane 3) under reducing conditions. Lane 4 shows molecular weight markers.

FIG. 2 shows the results of a time course for juvenile swine injected with either tetrameric recombinant BChE (PROTEXIA™-4MER-200 mg i.v.) or with the PEG-derivative of PROTEXIA™. Enzyme activity is measured in U/ml and time in hours.

### DEFINITIONS

The following defined terms are used throughout the present specification, and should be helpful in understanding the scope and practice of the present invention.

By “nerve agents” is meant substances, generally prepared by chemical synthesis or extraction from natural sources, that may cause deleterious or undesirable effects to a living creature if inhaled, absorbed, ingested, or otherwise encountered because of their high reactivity with and inhibition of cholinesterases, e.g., as discussed in the Background of the Invention. These agents include all of the agents discussed above, e.g., organophosphorus compounds, such as diisopropylfluorophosphate (DFP), CA (tabun), GB (sam), GD (soman), GE (cyclosarin), GE, CV, yE, VG (amiton), VM, VR (RVX or Russian VX), VS, VX, and combinations thereof. The foregoing list is exemplary and not limiting.

By “nerve agent poisoning” is meant deleterious or undesirable effects to a living creature exposed to a nerve agent or an organophosphorate pesticide. Organophosphate pesticides include acephate, azinphos-methyl, bensulide, cadusafos, chlorethoxyfos, chlorpyrifos, chlorpyrifos methyl, chlorthiophos, coumaphos, dialiflor, diazinon, diehlorvos (DDVP), dierotophos, dimethoate, dioxathion, disulfoton, ethion, ethoprop, ethyl parathion, fenamiphos, fenitrothion, fenthion, fonofos, isazophos methyl, isofenphos, malathion, methamidophos, methidathion, methyl parathion, mevinphos, monocrotophos, naled, oxydemeton methyl, phorate, phosalone, phosmet, phosphamidon, phostebupirim, pirimiphos methyl, profenofos, propetamphos, sulfotepp, sulprofos, temephos, terbufos, tetraehlorvinphos, tribufos (JDEF), trichlorfon. The foregoing list is exemplary and not limiting.

The phrase “therapeutically effective amount” is used herein to mean an amount sufficient to cause an improvement in a clinically significant condition in the host. For example, a therapeutically effective amount can be an amount sufficient to reduce by about 15 percent, preferably by about 50 percent, more preferably by about 90 percent, and most preferably prevent, a clinically significant deficit in the activity, function and response of the host.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness, and the like, when administered to a human. Preferably, as used herein, the term “pharmaceutically acceptable” means approved by a regulatory agency of the Federal or a state government or listed in the U.S. Pharmacopeia or other generally recognized pharmcopeia for use in animals, and more particularly in humans. The term “carrier” refers to a diluent, adjuvant, excipient, or vehicle with which the compound is administered. Such pharmaceutical caters can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “subject” as used herein refers to a mammal (e.g., rodent such as a mouse or rat, pig, primate, or companion animal, e.g., dog or cat, etc.). In a specific and non-limiting embodiment the term refers to a human.

The terms “about” and “approximately” mean within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to +1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

By “nerve agent neutralizing enzyme” is meant an enzyme capable of neutralizing or degrading nerve agents. These agents include all of the enzymes discussed in the background, e.g., cholinesterases, aryidialkylphosphatases, organophosphate hydrolases (OPH), carboxylesterases, triesterases, phosphotriesterases, arylesterases, paraoxonases, organophosphate acid anhydrases and diisopropylfluorophosphatases. In one embodiment, the present invention provides for the use of a cholinesterase. In another embodiment, the present invention provides for the use of butyrylcholinesterase. These nerve agent neutralizing enzymes may operate in a stoichiometric ratio, by binding and inactivating nerve agents in a 1:1 ratio. These nerve agent neutralizing enzymes may also operate by enzymatically cleaving nerve agents, and may inactivate nerve agents in a ratio of one nerve agent neutralizing enzyme to twenty or more nerve agent molecules.

By “cholinesterase” (ChE) is meant a family of enzymes involved in nerve impulse transmission. The major function of ChE enzymes is to catalyze the hydrolysis of the chemical compound acetylcholine at the cholinergic synapses. Electrical switching centers, called synapses, are found throughout the nervous systems of humans, other vertebrates and insects. Muscles, glands, and neurons are stimulated or inhibited by the constant firing of signals across these synapses. Stimulating signals are carried by the neurotransmitter acetylcholine, and discontinued by the action of ChE enzymes, which cause hydrolytic breakdown of acetylcholine. These chemical reactions occur continuously at a very fast rate, with acetylcholine causing stimulation and ChE enzymes ending the signals. The action of ChE allows the muscle, gland, or nerve to return to its resting state, ready to receive another nerve impulse if need be.

By “butyrylcholinesterase enzyme” or “BChE enzyme” is meant a polypeptide capable of hydrolyzing acetylcholine and butyrylcholine, and whose catalytic activity is inhibited by the chemical inhibitor iso-OMPA. Specific and non-limiting BChE enzymes to be produced by the present invention are mammalian BChE enzymes. Specific and non-limiting mammalian BChE enzymes include human BChE enzymes. The term “BChE enzyme” also encompasses pharmaceutically acceptable salts of such a polypeptide.

By “recombinant butyrylcholinesterase” or “recombinant BChE” is meant a BChE enzyme produced by a transiently transfected, stably transfected, or transgenic host cell or animal. The term “recombinant BChE” also encompasses pharmaceutically acceptable salts of such a polypeptide. Recombinant butyrylcholinesterase is well known in the art and is readily available (Arpagns et al, Biochemistry, 1990, 29:124-13 1; U.S. Pat. No. 5,215,909; Soreq et al., J. Biol. Chem., 1989, 264:10608-10613; Soreq et al., EMBO Journal, 1984, 3(6)1371-1375). In a specific and non-limiting embodiment, recombinant BChE is obtained in high yield from the milk or urine of transgenic animals (PCT Publication No. WO 03/054182).

The term “PEGylation” or just “pegylation” refers to use of polyethylene glycol (PEG or Poly(oxy-1,2-ethanediyl)-α-hydro-ω-hydroxy.) for coupling to the functional groups of biological molecules, such as proteins, antibodies and the like. Herein, the PEG is attached to a molecule that is a cholinesterase, for example, butyrylcholinesterase (BChE). The product of such pegylation varies depending on the reaction conditions, which in turn depend on the nature of the molecule to be pegylated, the specific pegylation site, the reagent used to pegylate and the extent of pegylation, which may depend both on the time of reaction and on the molar ratio of PEGs to substrate. The sites on proteins for such pegylation include: amine groups (both primary and secondary), thiol groups, and carboxyl groups. Useful PEGs are commonly activated prior to use in the pegylation procedure. Commonly used activated PEGs include those attached to maleimides and amines. Use of a specific activated group will commonly depend on the nature of the site to be pegylated.

### DETAILED DESCRIPTION OF THE INVENTION

The present invention provides pegylated therapeutic proteins, for example, pegylated butyrylcholinesterase (PEG-BChE), having improved clinical properties such as decreased dosage requirements, increased circulation time, enhanced solubility, sustained absorption and reduced immunogenicity.

Butyrylcholinesterase derived from human serum is a globular, tetrameric molecule with a molecular mass of approximately 340 kDa. Nine Asn-linked carbohydrate chains are found on each 574-amino acid subunit (or monomer). The tetrameric form of BChE is the most stable and is specific and non-limiting for therapeutic purposes. Wildtype, variant, and artificial BChE enzymes can be produced by those skilled in the art, such as by recombinant or chemo-synthetic means.

Preferably, the BChE enzyme utilized according to the method of the present invention comprises an amino acid sequence that is substantially identical to a sequence found in a mammalian BChE, for example, human BChE. In one embodiment, the BChE sequence is identical to human BChE. The BChE of the invention is typically be produced as a dimer or a monomer. In a specific and non-limiting embodiment, the BChE of the invention has a glycosylation profile that is substantially similar to that of native human BChE.

The amino acid sequence of wildtype human BChE is set forth in U.S. Pat. No. 6,001,625 to Broomfield, et al., which is hereby incorporated herein in its entirety. This patent also discloses a mutant human BChE enzyme in which the glycine residue at the 117 position has been replaced by histidine (identified as G117H). This mutant BChE has been shown to be particularly resistant to inactivation by organophosphate compounds [Lockridge, et al. Biochemistry (1997) 36:786-795]. Accordingly, this particular form of the BChE enzyme is especially useful for treatment of pesticide or war gas poisoning. Additional variants and mutants of BChE enzymes which may be produced according the methods of the present invention are disclosed in the U.S. Pat. No. 6,001,625.

Several methods are known in the art for introducing mutations within target nucleic acid sequences which may be applied to generate and identify mutant nucleic acid sequences encoding mutant BChE enzymes. Such mutant BChE enzymes may have altered catalytic properties, temperature profile, stability, circulation time, and affinity for cocaine or other substrates and/or certain organophosphate compounds.

The template nucleic acid sequences to be used in any of the described mutagenesis protocols may be obtained by amplification using the PCR reaction (U.S. Pat. Nos. 4,683,202 and 4,683,195) or other amplification or cloning methods. The described techniques can be used to generate a wide variety of nucleic acid sequence alterations including point mutations, deletions, insertions, inversions, and recombination of sequences not linked in nature. Note that in all cases sequential cycles of mutation and selection may be performed to further alter a mutant BChE enzyme encoded by a mutant nucleic acid sequence.

Mutations can be introduced within a target nucleic acid sequence by many different standard techniques known in the art. Site-directed in vitro mutagenesis techniques include linker-insertion, nested deletion, linker-scanning, and oligonucleotide-mediated mutagenesis (as described, for example, in “Molecular Cloning: A Laboratory Manual.” 2nd Edition” Sambrook, et al. Cold Spring Harbor Laboratory:1989 and “Current Protocols in Molecular Biology” Ausubel, et al., eds. John Wiley & Sons:1989). Error-prone polymerase chain reaction (PCR) can be used to generate libraries of mutated nucleic acid sequences (“Current Protocols in Molecular Biology” Ausubel, et al., eds. John Wiley & Sons: 1989 and Cadwell, et al. PCR Methods and Applications 1992 2:28-33). Altered BChE-encoding nucleic acid sequences can also be produced according to the methods of U.S. Pat. No. 5,248,604 to Fischer. Cassette mutagenesis, in which the specific region to be altered is replaced with a synthetically mutagenized oligonucleotide, may also be used [Arkin, et al. Proc. Natl. Acad. Sci. USA (1992) 89:7811-7815; Oliphant, et al. Gene (1986) 44:177-183; Hermes, et al. Proc. Natl. Acad. Sci. USA (1990) 87.696-700]. Alternatively, mutator strains of host cells can be employed to increase the mutation frequency of an introduced BChE encoding nucleic acid sequence (Greener, et al. Strategies in Mol. Biol. (1995) 7:32).

Various forms of the BChE (e.g., monomers, dimers and trimers) have demonstrated substrate activity and the pegylated forms of these are encompassed by the invention. In accordance with the invention, pegylated dimers and monomers of BChE are useful in treating such conditions as organophosphate poisoning, cocaine overdose and other diseases. For monomers and dimers of BChE, pegylation greatly improves their stability, giving them longer lifetimes in the system of an animal receiving such. Thus, pegylated monomers are satisfactory for the purposes of the invention and may, in some cases, be preferred.

PROTEXIA™ is a form of BChE formed using a β-Casein/hBChE transgene. This gene is used to generate transgenic animals and contains a dimerized chicken β-globin gene insulator (2.4 kb), a goat casein promoter, the β-casein gene up to and including the signal peptide sequence in exon 2, the human BChE cDNA gene sequence followed by a stop codon and a 6 kb fragment consisting of the β-casein coding and 3′-non-coding region. The methodology used to produce PROTEXIA™ is fully described in U.S. 2004/0016005 (22 Jan. 2004), U.S. Pat. No. 5,907,080 (25 May 1999) and U.S. Pat. No. 5,780,009 (14 Jul. 1998), the disclosures of all of which are hereby incorporated by reference in their entirety. In accordance with the present invention, PROTEXIA™ is a useful substrate for pegylation and the pegylated product is useful for treating conditions as disclosed herein, such as organophosphate poisoning, cocaine overdose and addition, as well as other maladies.

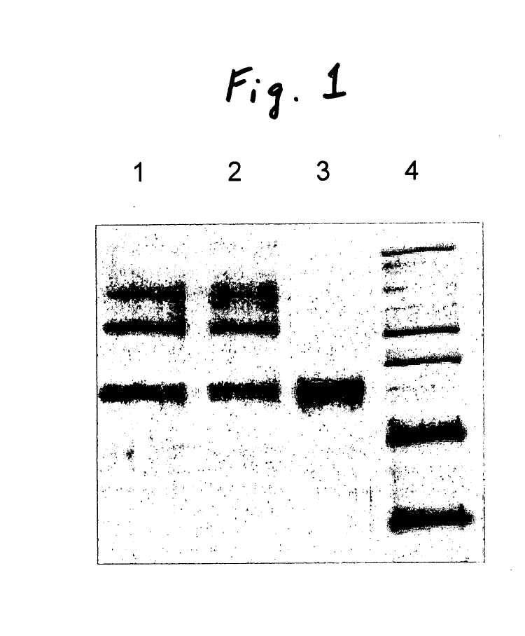

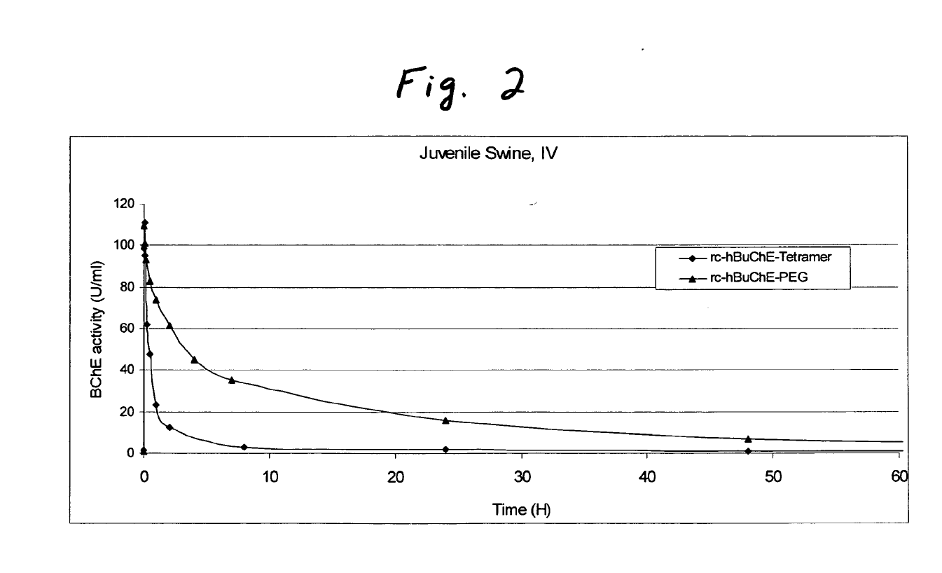

A specific activity of 720 U/mg, measured at 25° C. with 1 mM butyrylthiocholine in 0.1 M potassium phosphate, pH 8.0, was used as the standard for pure (i.e., 100%) human BChE. The resulting activity values for units/ml were converted to mg of active hBChE by using the relationship: 1 mg active hBChE=720 units. PROTEXIA™ was further subjected to modification by attachment of polyethylene glycol as described herein. A gel (SDS-PAGE) comparison of BChE with and without PEG attachment is shown in FIG. 1. The decreased migration on SDS-PAGE for the PEGylated form over the dimer with no modification is shown in FIG. 2.

In accordance with the present invention, human butyrylcholinesterase (hBuChE) has been shown to be effective in preventing organophosphate toxicity in several animal species. The availability of this enzyme in large quantities and its long circulatory stability are prerequisites for its widespread use as a bioscavenger in-vivo. This study evaluated the pharmacokinetics of a PEGylated form of transgenically produced recombinant hBuChe (PROTEXIA™). PROTEXIA™ purified from the milk of transgenic goats had a specific activity of approximately 700 u/mg (as measured by the Ellman assay) and migrated as a single band on SDS-PAGE under reducing conditions. Non-denaturing PAGE gels stained for activity with butyryl-thiocholine revealed that PROTEXIA™ secreted in the milk of transgenic goats consisted of a mixture of monomer, dimer and tetramer species with dimer being the predominant form. The mixture of these forms was either assembled into tetramers in-vitro (˜60-70% tetramer content) using poly-proline or subjected to PEGylation using standard techniques. Both preparations were injected i.v. into either rats, approximately 300 g, bw (n=4, 32 mg of PROTEXIA™) or juvenile swine, approximately 20 kg, bw (n=3, 200 mg of PROTEXIA™). Analysis of serial blood samples using the Ellman assay revealed a substantial enhancement of the MRT of the PEGylated Protexia™ preparation in both species when compared with the tetramer control:

TABLE 1

| Species | PROTEXIA ™ | MRT (hr) |
|---|---|---|
| Rat (4 animals) | Tetramer | 2 |
| | PEGylated | 15 |
| Juvenile Swine | Tetramer | 13 |
| (3 animals) | PEGylated | 36 |

For the above Table 1, rats weighed about 300 g each and each received a dose of 32 mg (i.v.) PROTEXIA™ while each juvenile swine weighed about 30 kg and each received 200 mg (i.v.) PROTEXIA™. A time course for the juvenile swine is shown in FIG. 2. In one embodiment, it was found that tetramerization of dimers using poly-L-proline did not significantly increased MRT over the dimer whereas pegylation of the dimer did significantly increase MRT versus the non-pegylated dimer or the tetramer formed from dimers using polyproline. These results suggest that PEGylation is an effective strategy for modulating the MRT of PROTEXIA™ in-vivo.

The recovered enzyme has purity of >98% and can be isolated from milk using tangential flow filtration, HQ anion exchange chromatography and affinity chromatography with Procainamide. Polyethylene glycol (PEG) is then conjugated to BChE using activated PEG reagents as described herein.

Linear monofunctional polyethylene glycol (PEG) is a polymer of ethylene units having the formula (CH₂CH₂O)ₙ—H that may be supplied commercially with a methoxyl group at the end (forming a monomethylether PEG). Only activated PEGs are useful in forming the derivatives of the invention. In addition, activated PEGs used in the invention should be as pure as possible, with as low a concentration as possible of impurities such as diols (which are potential cross-linking agents). Diols can be removed by ion exchange chromatography after first carboxylating the PEG. Such impurities should be removed prior to activation.

Because the PEGs are polymers, molecular weight is a consideration and PEGs with molecular weights of from about 5 kD to about 500 kD are most useful, with higher molecular weight PEGs still being of some value. For activated PEGs having multiple arms (such as forked PEGs), including anywhere from 2 to 8 arms, the linking centers for the PEGs may be any moiety of choice, such as derivatives of glycerine, for example, hexaglycerine to form an 8 arm PEG, or erythritol, for example, pentaerythritol to form a 4 arm PEG.

Pharmacokinetics of PEG-BChE has been studied in Guinea pigs: the half-life of recombinant BChE is less than two hours, while that of PEG-BChE is more than 40 hours. Further in accordance with the present invention, the bioavailability of recombinant BChE is less than 10% while that of PEG-BChE is 40-60%, in vitro efficacy tests show that PEG-BChE reacts with common nerve agents with the same efficiency as native BChE and in vivo efficacy tests shows that PEG-BChE works as efficiently as native BChE.

It should be borne in mind that PEGylation of BChE, by whatever reagent and/or strategy disclosed herein, may not result in a completely homogeneous product. Thus, fractionation to maximize the percentage of the principal PEGylated product(s) may be advantageous.

PEGs are readily soluble in a variety of organic solvents, such as acetone, dichloromethane, chloroform, ethyl acetate, acetonitrile, N,Ndimethylformamide(DMF), and water, all at room temperature but tend to be less soluble in solvents like methanol and ethanol, and are fairly insoluble in ether. The structure of a pegylated molecule, such BChE, can be determined by common methods used to study protein structure, such as sodium dodecyl sulfate polyacrylamide gel electrophoresis (SDSPAGE), mass spectroscopy, and high performance liquid chromatography (HPLC). Often, the protein product can be mapped to determined the location or site of the PEG attachment(s) and then reduced to fragments for analysis by liquid chromatography and mass spectroscopy.

PEGs for use in the present invention may be of different types, such as linear PEGs. The latter are straight-chained PEGs containing one or more functional groups, which may be the same or different from each other. For example, a linear monofunctional PEG has a reactive group at only one end, a linear homobifunctional PEG has the same reactive moiety at each end of the PEG and a linear heterobifunctional PEG has a different reactive group at each end of the PEG. Where it is desired to prevent reaction at one end of a PEG, this end may be bound to a chemically non-reactive group, such as a methoxy group.

PEGs useful in forming products of the invention may also be branched, which may contain 2 PEGs attached to a central core, from which extends a selected reactive group or may be a forked PEG having 2 reactive groups at one end. Multifunctional PEGs allow possible increase in efficiency of attached moieties, such as the BChE of the present invention, by permitting more than one BChE moiety to be attached to a single PEG.

PEGs useful in the reactions forming products of the present invention will commonly be those that are the most uniform, thereby having the smallest value of polydiversity (which is a measure of the broadness of the molecular weight distribution of the PEGs and is calculated from the ratio of the number average molecular weight (Mn) to the weight average molecular weight (Mw). A value of 1 means that these values are equal and the polymer is monodispersed. Typically, the PEGs useful in the present invention will have polydispersity values close to 1 (although this will almost always be greater than 1).

The average lifetime for PEG itself, when injected intravenously, may lie between a matter of minutes to up to 20 hours or more as molecular weight of the PEG increases. Renal clearance rate of PEGs is dependent on the glomerular filtration rate of the kidney. Short linear strands of PEG have a high clearance rate, but large linear PEGs, multi-arm PEGs, and PEGylated proteins tend to have a slower clearance rate. Methods for working with PEGs and pegylated proteins has been described in numerous publications, such as Harris, J. M. and Zalipsky, S., eds, Poly(ethylene glycol), Chemistry and Biological Applications, ACS Symposium Series 680 (1997); Veronese, F. and Harris, J. M., eds, “Peptide and protein PEGylation,” Advanced Drug Delivery Reviews (2002) 54(4):453-609; Harris, J. M. and Veronese, F. M., eds, “Peptide and Protein PEGylation II—clinical evaluation,” Advanced Drug Delivery Reviews (2003) 55(10): 1259-1350; Pasut, G., Guiotto, A. and Veronese, F. M., Expert Opin. Ther. Patents (2004) 14(5): 1-36.

In accordance with the foregoing, the present invention relates to a PEGylated butyrylcholinesterase (PEG-BChE) comprising a butyrylcholinesterase (BChE) protein chemically linked to polyethylene glycol (PEG). In a specific and non-limiting embodiment, the BChE is recombinant BChE, and in one embodiment transgenically-produced BChE, and most preferably wherein said BChE is chemically linked to said PEG by a covalent bond. In specific embodiments thereof, the PEG is attached to an amino group of said BChE, especially where said amino group is the N-terminal amino group of said BChE or said PEG is attached to a thiol group of said BChE, especially wherein said thiol group is on the N-terminal amino acid of said BChE, or where said PEG is attached to a glycan group of said BChE, especially where the PEG is attached to said glycan through a sialic acid group.

In other embodiments, the PEG has a linear structure or is has a branched or forked structure.

In an embodiment, the PEG is a member selected from the group consisting of mPEG-SPA, mPEG2-NHS and mPEG-MAL2.

In other embodiments, the PEG has a molecular weight of 5,000 to 500,000 kilodaltons.

Other examples include cases where a sample of the PEG-BChE of the invention, when administered to a mammal, has a half-life, or a mean residence time (MRT) in said mammal of at least 5 hours, more preferably at least 10 hours, more preferably at least 15 hours, more preferably at least 20 hours, or as long as at least 30 hours or 40 hours

Further specific and non-limiting embodiments include those wherein a sample of PEG-BChE, when administered to a mammal, has a bioavailability of at least 10%, more preferably at least 20%, more preferably at least 30%, still more preferably at least 40%, yet more preferably at least 50% and most preferably at least 60%.

Preferably, the PEG-BChE of the present invention contains PEG with an average molecular weight of about 20 kilodaltons.

In another example, the BChE protein used in the invention comprises the amino acid sequence of a mammalian BChE, especially wherein said mammal is a human being.

The present invention also relates to a method of preparing a PEG-BChE comprising contacting a BChE protein, for example, a recombinant monomer or dimer, with an activated PEG moiety under conditions promoting chemical linkage of said activated PEG to said BChE. In specific and non-limiting embodiments, said BChE is recombinant BChE or transgenic BChE and said activated PEG has a molecular weight of 5,000 to 500,000 daltons. Also specific and non-limiting is where the contacting occurs in a buffer having a pH of 4 to 11 and/or where the ratio of activated PEG to BChE protein (PEG:protein) is at least 2, more preferably wherein the ratio of activated PEG to BChE protein (PEG:protein) is between 2 and 500. For uses disclosed herein, a suitable ratio of activated PEG to BChE is about 80 to 1, which is found to produce a 1:1 ratio of PEG to BChE monomeric unit, with the product mostly dimers (thus, about 2 PEGs per dimer). In general, depending on the nature of the pegylating reagent that is employed, any ratio can be used so long as it does not detract from the biological activity of BChE.

In specific embodiments, the molecular weight of the activated PEG is reagents ranges from 5000 Dalton to 500,000 Dalton. In other specific and non-limiting embodiments, the coupling reaction is carried out in a buffer having a pH from 4 to 11, in one case pH 4 to 10, in another case pH 5 to 10, or pH 6 to 10, or pH 6 to 9, with pH values of about pH 6 or 7 or 8 or 9 being most advantageous. In the methods disclosed herein, the PEG:protein molar ratio in conjugation reaction is from 2 to 500, more specifically from 5 to 400, or from 10 to 300, or from 20 to 200 or from 30 to 100, or from 50 to 100, or from 60 to 90, or from 70 to 90. In one non-limiting example, a ratio of about 80:1 was used to generate PEG-BChE. Also in the methods of the invention, the temperature of the conjugation reaction is from, or from 10° C. to 40° C., or from 15° C. to 30° C., or about 20° C. to 25° C., with about 25° C. being advantageous. In addition, in the methods of the invention the conjugation reaction time is from 10 minutes to 24 hours. Also in the methods of the invention the protein concentration in the conjugation reaction is 0.1 to 10 mg/ml.

In other embodiments, the BChE is present at a concentration of at least 0.1 mg/ml, more preferably the BChE is present at a concentration of between 0.1 mg/ml and 10 mg/ml. Also specific and non-limiting is where the contacting occurs at a temperature of between 4° C. and 50° C. Further specific and non-limiting is where the sample of BChE proteins is contacted with a sample of activated-PEG moieties. In other specific and non-limiting embodiments the contacting is permitted to continue for at least 10 minutes, more preferably at least 24 hours.

In a further embodiment of these methods, the PEG-BChE is further purified using procainamide affinity chromatography or ion exchange chromatography. A drawback to use of procainamide is the possibility that it might be present in the final product, which is not desirable. Other methods, such as HPLC, may be more advantageous. It is to be noted that the method of purifying the final product in no way limits the nature or utility of the pegylated-BChE of the invention. Other methods useful in producing the PEG-BChE structures of the invention include use of different types of resins, for example, hydroxyapetite, ion exchange and special HPLC results, as well as affinity chromatography. In addition, use of the presence of the PEG moiety to facilitate purification is also within the skill of those in the art and finds use in the present methods. In general, one may proceed by removing water-sensitive materials, fractionating the pegylated products (based on size, such as separating monomers, dimers and tetramers), then proceed with the desired structure, for example, pegylated monomers, using resins and other procedures. This may then be followed by other procedures, such as preparative HPLC.

In purifying the pegylated butyrylcholinesterase (PEG-BChE) of the invention, may require up to 2 processing steps: purification of BChE and then purification of the PEG-BChE final product. In addition, scale-up will generally be required. Because purified BChE can already be obtained as described elsewhere herein, the process for obtaining PEG-BChE, or other pegylated proteins and peptides of the invention must be approached with foresight. In obtaining pegylated products of the invention, such as a pegylated protein, it is to be noted that pegylated proteins generally have a larger size and lower surface charge than the original native protein and samples of such product may well contain undesirable side products, a problem that may well affect the purification strategy (i.e., post-pegylation purification) as well as use of the products of the present invention for therapeutic purposes.

In addition, while a pure product is desirable, yield is also of concern because of the intended therapeutic use. For example, PEG-BChE finds its therapeutic value mostly in controlling and/or preventing the effects of toxic exposure. Thus, where PEG-BChE is to be used to ameliorate the effects of exposure to an organophosphate poison, the method necessarily involves reaction of a large molecule (PEG-BChE) with a small one (a small organic toxin) so that a large dose (say, several grams) of PEG-BChE may need to be administered to bolster the BChE that may already be present in the exposed victim. Thus, scale up considerations are important. There must be a weighing of purity versus yield, both of which must be optimized. In sum, larger amounts of material are desirable for uses contemplated herein.

Needless to say, purity may be a lesser consideration where treatment of a neurotoxic condition is to be achieved, since the effects of any impurities in the PEG-BChE may be of much less concern than the effects of the toxin to be nullified. In addition, because of the presence of the PEG, commonly used purification methods may be of little value, such as affinity columns that may rely on sites on BChE no longer available for such purposes due to the pegylation (although the active site of the BChE must be minimally affected by pegylation). Thus, techniques such as affinity chromatography, HPLC (high performance liquid chromatography), SEC (size exclusion chromatography), IEC (ion exchange chromatography), HIC (hydrophobic interaction chromatography), IEF (isolelectric focusing) and PAGE (polyacrylamide gel electrophoresis) will all likely be impacted by the presence of the PEGs on the protein molecule. Such methods are not only useful in purifying the products of the invention but may also be used to map the locations of PEG-bound sites within the protein, such as following tryptic digestion, or digestion with other endo- or exopeptidases.

Because pegylated proteins are very large molecules, the likely radius of the pegylated protein can be deduced from the molecular weight of the protein and that of the PEG used for conjugation. Such size effects may serve to separate native and pegylated products based on size exclusion (for example, using gel chromatography with resins like Sepharose or Superdex™ 200 and the like). In accordance with the present invention, where pegylated monomers of BChE are to be produced for use in the methods herein, gel chromatography (based on size exclusion) is a useful procedure for purifying the products of the invention.

Where ion exchange chromatography or isoelectric focusing is to be employed for purification, pegylation can affect isoelectric point (pI) so that pH values of elution buffers should be far from the pI values when loading. In addition, pI should be determined for the pegylated protein before use. Initial effluent should also be monitored to detect any loss of initial sample. In all such procedures, use of step gradients can be more effective than linear gradients in obtaining high yields of product.

Pegylated BChE has been produced herein to high purity and with long survival times in plasma (see Table 1). Of course, different PEG-derivatives of BChE will have different MRT values and one can easily utilize these to determines MRTs as high as 60 hours or beyond. In producing the pegylated derivatives of the invention, having high MET values, it is to be noted that there are specific and non-limiting sites for pegylation of the BChE molecules, which can readily be determined by dissecting the molecule after pegylation and then relating the extent and location of PEG moieties with the observed MRT values of different derivatives. Herein, it is to be noted that variations occurred for varying lysine derivatization (any combination of the some 40 lysines present in BChE) so that there are specific and non-limiting lysines to be pegylated within the BChE protein, which selected pegylation results in prolonged MRT values. In accordance with the present invention, the highest MRTs were observed in guinea pigs receiving pegylated-BChE having one PEG per subunit and attached to a lysine residue.

Pegylated BChE structures produced by the methods of the invention and useful in methods described herein may be in the form of a monomer, as well as a dimer. Such monomers may possess one or more than one PEGs per monomer, with one PEG per monomer being one specific embodiment. Use of such pegylated monomers is a specific embodiment of the invention, which specifically contemplates production of BChE by recombinant means, which methods are especially conducive to production of monomeric (i.e., single polypeptide chain) products with no requirement for formation of intermolecular disulfide bonds or assembly of the monomers into supramolecular structures, although dimers may also be present in compositions of the invention.

In other embodiments, the chemical linkage is to an amino group on said BChE protein, more preferably the activated PEG is a mono-functional methoxy-activated polymer of succinimidyl derivatives. In specific embodiments thereof, the succinimidyl derivative is a member selected from the group consisting of succinimidyl propionic acid (mPEG-SPA), α-methylbutanoate (mPEG-SMB) and N-Hydroxysucciminidyl (mPEG-NHS). Also specific and non-limiting is where the amino group is the N-terminal amino group.

In other specific and non-limiting embodiments of such methods, the activated PEG is a mono-functional methoxy-activated polymer bearing one or more aldehyde groups, preferably wherein said mono-functional methoxy-activated polymer is Butyraldehydyl-PEG (PEG-ButyrALD). In other such embodiments said chemical linkage is to a thiol group on said BChE protein, preferably wherein said activated PEG is Maleimide-coupling-PEG (mPEG-MAL), or where the thiol group is on the N-terminal amino acid of said BChE protein. In specific and non-limiting embodiments, the activated PEG is a mono-functional methoxy-activated PEG, or is mPEG-OPTE.

In another specific and non-limiting embodiment, the chemical linkage is to a glycan group on said BChE, such as where the activated-PEG is linked to sialic acid. Activated PEGs may be purchased commercially.

Where the PEG is to be attached to an amino group of the BChE, the PEG may be activated with electrophilic groups. Useful activated derivatives of PEG for such protein groups include the N-hydroxysuccinimide (NHS) ester. Thus, reaction between the epsilon amino group of lysine(s) or the N-terminal amine and the NHS ester produces a physiologically stable amide linkage(s). The resulting monofunctional polymers may be capped on one end by a methoxy group (mPEG) and produce products free of cross-linking. Use of such PEG-NHS activated esters is advantageous because the coupling with the target protein, here BChE, can be accomplished at physiological pH. However, change in pH, temperature and length of reaction may also help to determine which of the lysines on the target react with the activated PEG.

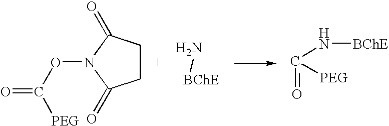

Succinimidyl-α-methylbutanoate is an α-methyl substituted PEG that provides a sterically hindered active ester for reaction with amino groups on proteins, such as BChE, and may result in increased hydrolytic stability of the activated ester due to greater stability of the resulting amide linkage. More importantly, the activated ester is less reactive and may thereby afford greater target selectivity during reaction with BChE (i.e., selectivity in terms of the particular amino group attacked). Further, steric hindrance provided by the α-methyl group may slow enzymatic degradation in the subject to be treated with the PEG-BChE. Such a reagent has the following structure and forms the indicated derivative with BChE:

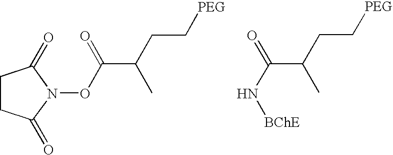

Reagents such as PEG-succinimidyl propionate are esters used in the PEGylation of amine functional groups to provide a physiologically stable amide linkage. The activated reagent plus BChE derivatives are as follows:

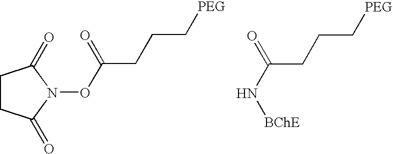

Also useful is the branched reagent PEG N-Hydroxysuccinimide, a high molecular weight monofunctional compound that can provide steric bulky and attach multiple PEGs to a single site. This reagent also has the property that it behaves as if it were larger than a corresponding linear PEG of the same MW while the compound is purely monofunctional. The resulting PEG-BChE may thereby experience greater in vivo stability and longer MRT because of greater resistance to degradative reactions and processes. In addition, such derivatives may exhibit greater resistance to pH degradation with reduced antigenicity and likelihood of triggering an immunological response. In addition, due to the bulkiness of the ligand, the resulting protein conjugate may greater enzymatic activity since it is unlikely that such a larger structure could enter the active site or compete with a much smaller organic structure for the active site of BChE. Again, the larger steric effect of this bulky radical can slow reaction with the protein and thereby afford greater selectivity of the reactive group (so that not all exposed amines will be tied up by the PEG. The structure of such a branched reagent and corresponding BChE derivative are as follows:

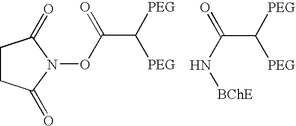

PEGs attached to aldehyde groups are reactive with primary amines through reductive amination using a reducing agent (for example, sodium cyanoborohydride). Such reagents react only with amines under mild conditions. However, many such reagents can present problems for pegylation of proteins, due partly to instability of the reagent. Such problems can be overcome by use of selected pegylating reagents. Such reagents are available commercially, for example, PEG-butyraldehyde reagents, which are more selective and stable at neutral pH. The pKa for N-terminal amines is lower than that for lysine or arginine side chains and such reagents are useful for selective modification of the N-terminus of proteins such as BChE. One such activated PEG has the structure: PEG-(CH₂CH₂CH₂COOH)ₙ wherein n=1 or 2. Branched structures may also be used, wherein two PEGs are attached, via a common moiety, to the γ-carbon of a single butyraldehyde group. The structures for a reagent and corresponding BChE derivative are as follows:

PEG-CH₂CH₂CH₂CHO PEG-CH₂CH₂CH₂CH₂—NH-BChE

Where the group to be pegylated is one of the thiol groups of BChE, several reagents are available to attach to such groups. One example of a reagent useful with the present invention is the maleimide derivative of PEG wherein the latter is attached to the nitrogen of the maleimide ring system. The structure of such a reagent and the corresponding BChE derivative are as follows:

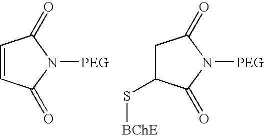

As shown, coupling of the maleimide to a thiol group of BChE (in general, a reaction highly specific for thiol groups) forms the 3-thiosuccinimidyl ether linkage, thereby attaching the PEG to the BChE. Such reactions often occur at neutral pH, which is useful for maintaining the native structure of the protein. In addition, because there are fewer thiol groups on BChE than amino groups the resulting product may be more selective and uniform in structure.

Such activated reagents may also be in the form of branched structures with two PEGs linked via a common moiety with a single maleimide system or wherein 2 maleimides are attached to a single PEG (a forked structure) or are attached to 2 PEGs having a structure:

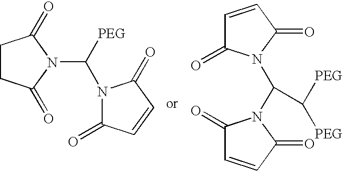

In another embodiment, the activated reagent comprising PEG attached to an ortho-pyridyldisulfide, via the disulfide group, affords a disulfide bond with a cysteine on BChE. Here, the o-pyridyldisulfide group is thiol-specific for free sulfhydryls under both acidic and basic conditions (pH 3-10) and oxidatively couples to a free sulfhydryl group on the BChE molecule. This linkage, although stable, is also reversible if introduced into a reducing environment (for example, dithiothreitol or mercaptoethanol) to afford the original free sulfhydryl group. Other advantages include release of pyridine-2-thione, a nonreactive compound that avoids further disulfide contamination, which release is readily monitored by increased absorbance at 343 nm. A useful reagent would have the structure:

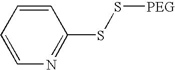

In accordance with the invention, a useful reagent also includes a single PEG attached to two pyridyldisulfide moieties for attached to 2 BChE molecules. Useful reagents for practice with the invention also include PEG attached to one or two simple thio —SH groups for thiol-specific pegylation of free thiols forming and forming a disulfide-bridged polymer conjugate to the cysteine side chain of BChE protein. Because there are fewer cysteines in BChE than there are side chain amino groups, greater control over location of the bound PEG can be achieved.

It should be borne in mind that in using multifunctional PEG derivatives, these need not have the same moieties in each case. Thus, a PEG attached to two different activating moieties is completely within the scope of the present invention so long as the reaction conditions permit both moieties to function in binding to the target protein. It should also be noted that for use with BChE, it is typically contemplated that only a single PEG will attach to a single BChE but the invention is not necessarily limited to that embodiment and thus bifunctional reagents, which would bind more than a single BChE to a given PEG, may yet find use in the methods of the invention. Such heterobifunctional PEGs are commercially available.

PEG amines (having the structure PEG-NH₂) also find use as reagents in the invention. Such use is contemplated in one aspect where the fact that BChE is a glycoprotein and such amino groups are highly reactive with sugar moieties on BChE (see, for example, Urrutigoity et al, Biocatalysis 2, 145 (1989)).

In all cases, the quantity and distribution of PEG moieties on the target protein, such as BChE, can be determined are determined by SEC-HPLC or by SDS-PAGE, as well as other techniques well known to those skilled in the art. Such methods as SEC-HPLC can be used not only to determine the extent of pegylation of a target moiety, like BChE, but also as a quantitative chromatographic method to demonstrate uniformity of pegylation between synthetic preparations (i.e., the consistency from one batch to another).

Pegylation may also be used to modify other catalytic molecules or those developed by targeted evolution methods, such as where error prone E. coli Pol I is used to produce DNA for cloning (i.e., Pol I containing mutations in is the domains controlling fidelity of replication).

The BChE-PEG agents of the present invention are intended for systemic administration, preferably by injection, but may also be administered by other routes, such as inhalation, where an inhalation device may be employed.

A nerve agent neutralizing enzyme as described herein can be present as part of a pharmaceutical composition. A pharmaceutical composition comprises a nerve agent neutralizing enzyme in combination with one or more pharmaceutically or physiologically acceptable carriers, diluents, or excipients. Such compositions may comprise buffers (e.g., neutral buffered saline or phosphate buffered saline), carbohydrates (e.g., glucose, mannose, sucrose or dextrose), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate.

Carrier suitable for use in the present invention may contain minor amounts of additives such as substances that enhance isotonicity and chemical stability of the pegylated protein and such materials are commonly non-toxic to recipients at the dosages and concentrations employed herein. These may include buffers such as phosphate, citrate, succinate, acetate, or other organic acids and/or salts thereof, as well as antioxidants such as ascorbic acid (Vitamin C), low molecular weight (less than about 8 to 10 residues) peptides, e.g., polyarginine or tripeptides, and also proteins, such as human serum albumin, bovine serum albumin, gelatin, or even antibodies, and also hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium, potassium, calcium, magnesium, and the like; and/or nonionic surfactants such as polysorbates, poloxamers, and certain detergents.

Nerve agent neutralizing enzyme formulations suitable for use in the present invention include dry powders, solutions, suspensions or slurries, and particles suspended or dissolved within a propellant.

The nerve agent neutralizing enzyme compositions of the present invention may be combined with pharmaceutically acceptable excipients, including, but not limited to: (a) carbohydrates, e.g., monosaccharides such as fructose, galactose, glucose, D-mannose, sorbose, and the like; disaccharides, such as lactose, trehalose, cellobiose, and the like; cyclodextrins, such as 2-hydroxypropyl-.beta.-cyclodextrin; and polysaccharides, such as raffinose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamin hydrochloride, and the like; (d) peptides and proteins such as aspartame, human serum albumin, gelatin, and the like; and (e) alditols, such as mannitol, xylitol, and the like. A specific and non-limiting group includes lactose, trehalose, raffinose, maitodextrins, glycine, sodium citrate, human serum albumin and mannitol.

The amount of nerve agent neutralizing enzyme to be administered will be that amount necessary to deliver a therapeutically effective amount of the nerve agent neutralizing enzyme to achieve the desired result. In practice, this will vary widely depending upon the particular nerve agent neutralizing enzyme, the severity of the condition, the weight of the subject, and the desired therapeutic effect. In practice, the dose of nerve agent neutralizing enzyme may be delivered in one or more doses.

The nerve agent neutralizing enzyme compositions of the present invention may be suspended, dispersed, or dissolved in solution. The liquid carrier or intermediate can be a solvent or liquid dispersive medium that contains, for example, water, ethanol, a polyol (e.g. glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants. Prevention of the action of microorganisms can be achieved by the addition of various antibacterial and antifungal agents, e.g., paraben, chlorobutanol, or sorbic acid. In many cases isotonic substances are recommended, e.g. sugars, buffers and sodium chloride to assure osmotic pressure similar to those of body fluids, particularly blood.

In another aspect, the present invention relates to compositions of any of the compounds of the invention, preferably wherein such compound is present in a pharmaceutically acceptable carrier and in a therapeutically effective amount. Such compositions will generally comprise an amount of such compound that is not toxic (i.e., an amount that is safe for therapeutic uses). The present invention is thus drawn to a pharmaceutical composition comprising the PEG-BChE as disclosed herein in a pharmaceutically acceptable carrier, wherein said PEG-BChE is present in an amount effective to neutralize a toxin or poison. In a specific and non-limiting embodiment, this composition further comprises non-PEGylated BChE.

Sterile solutions can also be prepared by mixing the nerve agent neutralizing enzyme formulations of the present invention with an appropriate solvent and one or more of the aforementioned excipients, followed by sterile filtering. In the case of sterile powders suitable for use in the preparation of sterile injectable solutions, preferable preparation methods include drying in vacuum and lyophilization, which provide powdery mixtures of the isostructural pseudopolymorphs and desired excipients for subsequent preparation of sterile solutions.

Appropriate dosages and the duration and frequency of administration will be determined by such factors as the condition of the patient, the type and severity of the patient's disease and the method of administration. In general, an appropriate dosage and treatment regimen provides the nerve agent neutralizing enzyme in an amount sufficient to provide therapeutic and/or prophylactic benefit. Various considerations for determining appropriate dosages are described, e.g., in Goodman and Gilman, The Pharmacological Basis of Therapeutics, 1980, MacMillan Publishing Co, New York.

Appropriate dosages may also be determined using experimental models and/or clinical trials. In general, the use of the minimum dosage that is sufficient to provide effective therapy is specific and non-limiting. Patients can be monitored for therapeutic effectiveness using physical examination, imaging studies, or assays suitable for the condition being treated or prevented, which will be familiar to those of ordinary skill in the art. Dose adjustments can be made based on the monitoring findings. For example, an individual with exposure to nerve agent, following administration of nerve agent neutralizing enzyme according to the invention, for cessation of symptoms caused by the nerve agent. Based upon the foregoing considerations, determination of appropriate dosages will require no more than routine experimentation by those of ordinary skill in the art.

Methods of treatment contemplated using therapeutics such as PEG-BChE of the present invention include intravenous (IV) administration, intramuscular (IM) administration and administration using a patch that may last up to a month. The latter is especially useful for prophylactic purposes where possible exposure to toxic agents is anticipated but no specific time frame can be ascertained (for example, persons (such as soldiers) entering a warring theater or sent to investigate possible sources of toxins and wherein time for removal from such areas is initially indeterminate). Prior to administration such agent (for example, a PEG-BChE of the present invention) may be kept as a lyophilized powder, ready for mixing with a suitable carrier, excipient or diluent, such as water (distilled or not), a buffer, such as PBS, or some other pharmaceutically suitable solvent or suspending agent. Such formulations may or may not be sterile. In determining appropriate mixing, consideration must be given not only to therapeutically acceptable and effective carriers but also to concerns about solubility, which may be somewhat different for the pegylated protein versus the native protein. The Handbook of Pharmaceutical Excipients is a good source for such materials. Also to be considered are issues of stability. Thus, a formulation for a product of the invention, such as PEG-BChE, must be stable for varying amounts of time. Thus, where, for example, PEG-BChE is to be maintained in a hospital or other clinical environment for use as needed and to be administered by clinical staff, the PEG-BChE may be maintained as a lyophilized powder that can then be reconstituted for use as needed. Here, such carriers as PBS (phosphate buffered saline) are convenient. Alternatively, where PEG-BChE is to be carried by personnel into potentially dangerous areas, and then used as required, reconstitution may be inadequate to treat potential exposures to toxic agents. In such cases, the PEG-BChE may need to be maintained in a suspended state with the carrier already present, such as in a syringe carried in a sterile contained, for immediate use by a subject in need (such as immediately following known or suspected exposure to a toxic agent).

In a specific embodiment, the dosage is administered as needed. One of ordinary skill in the art can readily determine a volume or weight of nerve agent neutralizing enzyme formulation corresponding to this dosage based on the concentration of nerve agent neutralizing enzyme in a formulation of the invention, In another embodiment of the present invention, additional dosages may be administered if normal physiological functions have not been restored.

The present invention also relates to a method of neutralizing a toxin or poison in an animal, comprising administering to said animal an effective amount of a PEG-BChE pharmaceutical composition of the invention, preferably wherein said animal is a mammal, most preferably wherein said mammal is a human being. Also specific and non-limiting is where the toxin or poison is a toxin or poison that acts on the nervous system, including a C-series nerve agent, a V-series nerve agent or is an organophosphate. Also specific and non-limiting is where the toxin or poison is a member selected from the group consisting of diisopropylfluorophosphate (DFP), GA (tabun), GB (sarin), GD (soman), CF (cyelosarin), GE, CV, yE, VG (amiton), VM, VR (RVX or Russian VX), VS, and VX.

The PEG-derivatives of BChE disclosed according to the invention may be used in the treatment of a mammal, such as a human, for poisoning, for example, with an organophosphate agent or may be utilized prophylactically, where said mammal is likely to become exposed to such an agent. Because the compositions of the invention comprise BChE derivatives with high MRTs, they can be administered well in advance, such as days ahead of time, of an expected exposure. Other applications include any wherein BChE administration, or that of some other catalytic entity, such as some other cholinesterase, or some other enzyme or catalytic agent, or even other proteins and peptides, can prevent or treat a clinical condition, for example, individual conditions such as cocaine overdose and insecticide, for example, organophosphate, poisoning, or long-term illness, such as Alzheimer's disease, and other such afflictions. These can likewise be treated to cure or to prevent the effects of such maladies.

In a further specific and non-limiting embodiment, the wherein said pharmaceutical composition further comprises, or is administered in conjunction with, an agent selected from the group consisting of a carbamate, an anti-muscarinic, a cholinesterase reactivator and an anticonvulsive, preferably wherein said carbamate is pyridostigmine, or wherein said anti-muscarinic is atropine, or where the cholinesterase reactivator is pralidoxime chloride (2-PAM, Protopam). In another specific and non-limiting embodiment, the anticonvulsive is diazepam.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1.-70. (canceled)

71. A stable butyrylcholinesterase (PEG-BChE) comprising a recombinant butyrylcholinesterase (rBChE) protein covalently linked to polyethylene glycol (PEG) at a thiol group of said rBChE.

72. The stable PEG-BChE of claim 71, wherein said stable PEG-BChE is present as a rBChE-dimer having a single PEG attached to each monomeric subunit of said dimer.

73. The stable PEG-BChE of claim 72, wherein said rBChE protein was produced by a trangenic non-human mammal.

74. The stable PEG-BChE of claim 73, wherein said mammal is a goat.

75. The stable PEG-BChE of claim 72, wherein said PEG has a linear structure.

76. The stable PEG-BChE of claim 72, wherein said PEG has a branched or forked structure.

75. The stable PEG-BChE of claim 72, wherein said PEG is mPEG-MAL2.

76. The stable PEG-BChE of claim 72, wherein said PEG has a molecular weight of 5,000 to 500,000 kilodaltons.

77. The stable PEG-BChE of claim 72, wherein a sample of said PEG-BChE, when administered to a mammal, has a half-life in said mammal of at least 5 hours.

78. The stable PEG-BChE of claim 72, wherein a sample of said PEG-BChE, when administered to a mammal, has a half-life in said mammal of at least 20 hours.

79. The stable PEG-BChE of claim 72, wherein a sample of said PEG-BChE, when administered to a mammal, has a half-life in said mammal of at least 40 hours.

80. The stable PEG-BChE of claim 72, wherein a sample of said PEG-BChE, when administered to a mammal, has a bioavailability of at least 10%.

81. The stable PEG-BChE of claim 72, wherein a sample of said PEG-BChE, when administered to a mammal, has a bioavailability of at least 30%.

82. The stable PEG-BChE of claim 72, wherein a sample of said PEG-BChE, when administered to a mammal, has a bioavailability of at least 60%.

83. A method of preparing a stable PEG-BChE of claim 72, comprising contacting a rBChE protein with an activated PEG moiety under conditions promoting chemical linkage of said activated PEG to said rBChE, wherein the ratio of activated PEG to rBChE protein (PEG:protein) is between 40:1 and 120:1.

84. The method of claim 83, wherein the ratio of activated PEG to BChE protein (PEG:protein) is about 80:1.

85. The method of claim 83, wherein said activated PEG is Maleimide-coupling-PEG (mPEG-MAL).

86. A pharmaceutical composition comprising a stable PEG-BChE of claim 72 in a pharmaceutically acceptable carrier, wherein said PEG-BChE is present in an amount effective to neutralize a toxin or poison.

87. The pharmaceutical composition of claim 86, wherein said dimer of claim 2 makes up at least 80% of the PEG-BChE present in said composition.

88. The pharmaceutical composition of claim 87, wherein said PEG-BChE is a mixture of dimers and moomers.

89. The pharmaceutical composition of claim 86 or 87, wherein said composition was formed by reconstituting a lyophilized stable PEG-BChE of claim 2.

90. A method of neutralizing a toxin or poison in mammal, comprising administering to said mammal an effective amount of the pharmaceutical composition of claim 86, 87, 88 or 89.

91. The method of claim 90, wherein said mammal is a human being.

92. The method of claim 90, wherein said toxin or poison is a toxin or poison that acts on the nervous system.

93. The method of claim 90, wherein said toxin or poison is an organophosphate.

94. The method of claim 90, wherein said toxin or poison is a member selected from the group consisting of diisopropylfluorophosphate (DFP), GA (tabun), GB (sarin), GD (soman), CF (cyelosarin), GE, CV, yE, VG (amiton), VM, VR (RVX or Russian VX), VS, and VX.

95. The method of claim 90, wherein said pharmaceutical composition further comprises, or is administered in conjunction with, an agent selected from the group consisting of a carbamate, an anti-muscarinic, a cholinesterase reactivator and an anticonvulsive.
